(19) United States
(12) Patent Application Publication
(10) Pub. No.: US 2019/0223722 A1
(43) Pub. Date: Jul. 25, 2019

(54) Smart telehealth ECG recording system

(71) Applicant: VivaLnk, Inc., Santa Clara, CA (US)

(72) Inventor: Cecilia Xi, San Jose, CA (US)

(21) Appl. No.: 15/880,445

(22) Filed: Jan. 25, 2018

(51) Int. Cl. A61B 5/00; A61B 5/0468; A61B 5/0408

(52) U.S. Cl. CPC A61B 5/0006; A61B 5/04087; A61B 5/0468; A61B 5/6833

(57) ABSTRACT

A smart telehealth ECG recording system includes a wearable patch to be worn on a user's skin. The wearable patch includes a stretchable substrate, a pair of electrodes mounted in the stretchable substrate and configured to measurement a voltage signal across the user's skin to produce ECG data, a mechanical sensor configured to measure a pulse from the user's body to produce pulse data, an acceleration sensor configured to measure a movement of the user to produce movement data, an antenna over the stretchable substrate and in electric communications with the pair of electrodes, the mechanical sensor, and the acceleration sensor, wherein the antenna is configured to wirelessly transmit the ECG data, the pulse data, and the movement data associated with the user, and a circuit electrically connected with the pair of electrodes, the mechanical sensor, the acceleration sensor, and the antenna.

### BACKGROUND OF THE INVENTION

The present application relates to wearable electronic devices, and in particular, to wearable patches that can record electrocardiogram (ECG) signals and wireless transmit ECG data.

Electronic patches can be used for tracking objects and for performing functions such as producing sound, light or vibrations, and so on. As applications and human needs become more sophisticated and complex, electronic patches are required to perform a rapidly increasing number of tasks. Electronic patches are often required to be conformal to curved surfaces, which in the case of human body, can vary overtime. Electronic patches can communicate with smart phones and other devices using Wi-Fi, Bluetooth, Near Field Communication (NFC), Bluetooth, and other wireless technologies.

Wearable patch (or tag) is an electronic patch to be worn by a user. A wearable patch is required to stay on user's skin and operate for an extended period from hours to months. A wearable patch can contain a micro-electronic system can be accessed using NFC, Bluetooth, Wi-Fi, or other wireless technologies. A wearable patch can include different sensors for measurements such as vital signs monitoring.

Cardiac diseases especially arrhythmias can be detected using the ECG. However, most conditions are not sustaining and cannot be easily captured by a standard 10 sec ECG. In addition, the problematic episodes can be short and already be gone before the patient starts to feel the symptoms, or some patients may even be asymptomatic. Therefore, taking ambulatory ECG recordings are important for cardiac patients and patients with risks or associated symptoms, for screening, monitoring or drug titration.

Holter ECG is the option for standard at-home use, but it is inconvenient and cumbersome with wires and the recording box. With the wireless patch version, recordings are sent to the doctor for offline analysis after the monitoring period is over. There is no prompt data sharing or analysis. The patient cannot get prompt feedback or guidance for action. Most of Holter systems can conduct 24 hours to 72 hrs of monitoring, which is not long enough.

There are a few handheld ECG devices available in the market to use at home for short ECG recordings and sharing. These devices need the patient to initiate a recording with fingers from both hands to be put on the electrodes, wait for the settling time of the ECG (typically within 30 sec) and the recording is typically no longer than 100 seconds.

There is therefore a need for wireless smart system that can do long-term recordings and prompt data sharing and analysis.

### SUMMARY OF THE INVENTION

The presently application discloses a system or networked wearable patch(es) that can measure and monitor a person's vital signs and other signals, and a wireless control device that communicate with the networked wearable patch(es), and can dynamically control the measurement functions. The present disclosure teaches a wireless wearable patch suitable for recording ECG signals, and measuring a user's pulse and movement. The disclosed wireless smart system can conduct long-term recordings and prompt data sharing and analysis.

Moreover, the disclosed wearable patch is easy and comfortable to wear and do not require wire connections to heavy equipment. The measurements can be performed while a person fulfills his or her normal daily activities.

In one general aspect, the present invention relates to a smart telehealth ECG recording system that includes a wearable patch configured to be worn on a user's skin, which includes a stretchable substrate; a pair of electrodes mounted in the stretchable substrate and configured to measurement a voltage signal across the user's skin to produce ECG data; a mechanical sensor that can measure a pulse from the user's body to produce pulse data; an acceleration sensor that can measure a movement of the user to produce movement data; an antenna over the stretchable substrate and in electric communications with the pair of electrodes, the mechanical sensor, and the acceleration sensor, wherein the antenna can wirelessly transmit the ECG data, the pulse data, and the movement data associated with the user; and a circuit electrically connected with the pair of electrodes, the mechanical sensor, the acceleration sensor, and the antenna. A wireless control device can wirelessly receive the ECG data, the pulse data, and the movement data from the antenna, wherein the ECG data is analyzed based on the pulse data and the movement data.

Implementations of the system may include one or more of the following. The wearable patch can further include a semiconductor chip in connection with the circuit and configured to produce electric signals comprising the ECG data, the pulse data, and the movement data, which enables the antenna to wirelessly transmit the ECG data, the pulse data, and the movement data. The wearable patch can further include a circuit substrate comprising the circuit and on the stretchable substrate, wherein the semiconductor chip is mounted on the circuit substrate. The wireless control device can further include a measurement controller that can wirelessly transmit a measurement control signal to the antenna, wherein the semiconductor chip can measure ECG data, the pulse data, and the movement data in response to the measurement control signal. The measurement controller can control the semiconductor chip to vary a type, timing, a frequency, or duration of the ECG measurement, the pulse measurement, and the movement measurement. The smart telehealth ECG recording system can further include two rechargeable batteries that can supply power to the circuit and the semiconductor chip, wherein at least one of the two rechargeable batteries is removable to be charged. The wearable patch can further include a third electrode configured to be in contact with the user's skin, wherein the third electrode is in connection with the circuit and is configured to provide grounding to the circuit. The stretchable substrate can include an opening, wherein at least one of the pair of electrode comprises an electric conductive cup that is connected to the circuit and mounted in the opening in the stretchable substrate. The wireless control device can further include an intelligent analyzer that can analyze the ECG data based on the pulse data and the movement data to produce an analysis result. The wearable patch can further include an elastic layer formed on the stretchable substrate, the circuit substrate, and the pair of electrodes.

In another aspect, the present invention relates to a wearable patch configured to be worn on a user's skin, which includes a stretchable substrate; a pair of electrodes mounted in the stretchable substrate and configured to measurement a voltage signal across the user's skin to produce ECG data; a mechanical sensor that can measure a pulse from the user's body to produce pulse data; an acceleration sensor that can measure a movement of the user to produce movement data; an antenna over the stretchable substrate and in electric communications with the pair of electrodes, the mechanical sensor, and the acceleration sensor, wherein the antenna can wirelessly transmit the ECG data, the pulse data, and the movement data associated with the user to a wireless control device; and a circuit electrically connected with the pair of electrodes, the mechanical sensor, the acceleration sensor, and the antenna.

These and other aspects, their implementations and other features are described in detail in the drawings, the description and the claims.

### BRIEF DESCRIPTION OF THE DRAWINGS

FIG. 1 illustrates a smart telehealth ECG recording system including a wireless control device and a wearable patch attached to a user's body.

FIG. 2 is a system block diagram for the wireless control device in wireless communications of the wearable patch in accordance with some embodiments of the present invention.

FIG. 3 is a cross-sectional view of an exemplified wearable patch suitable for the disclosed smart telehealth ECG recording system in accordance with some embodiments of the present invention.

### DETAILED DESCRIPTION OF THE INVENTION

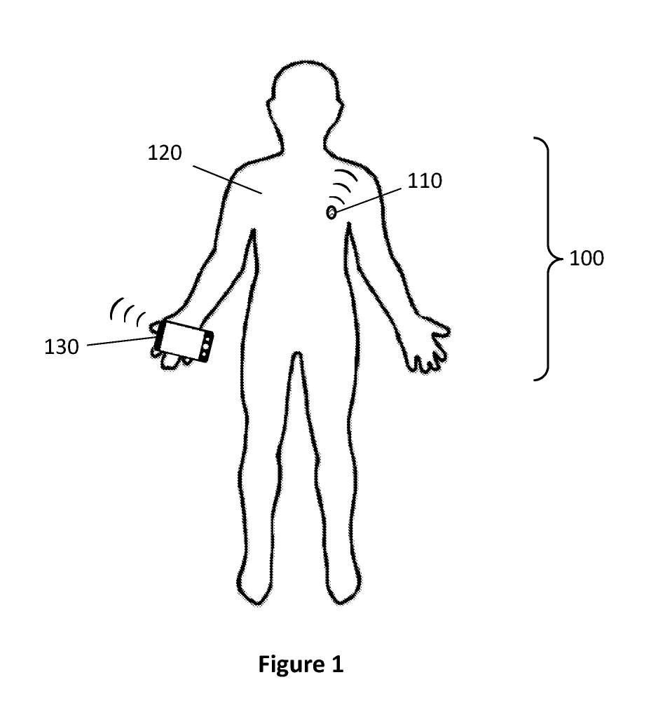

Referring to FIG. 1, a smart telehealth ECG recording system 100 includes a wearable patch 110 attached to the body or the skin of a user 120 for recording ECG and for measuring other body vital signs such as pulse, movement, etc. In the present disclosure, the term “wearable patch” can also be referred to as “wearable sticker”, “wearable tag”, or “wearable band”, etc.

The smart telehealth ECG recording system 100 also includes a wireless control device 130 that can wirelessly exchange data with the networked wearable patch 110. The wireless communications can be conducted using Wi-Fi, Bluetooth, Near Field Communication (NFC), and other wireless technologies. The wireless control device 130 can be a portable mobile device, which the user 120 can carry with him or her. The wireless control device 130 can also be a stationary device that can be placed at home or office where the user 120 may stay for an extended period. The portable mobile device can be implemented with specialized hardware and software units built in a smart phone, smart watch, a tablet computer (including devices such as iPod), or a dedicated health or sport monitoring device. The wireless control device 130 can be in communication with a network server in which a user account is stored for the user.

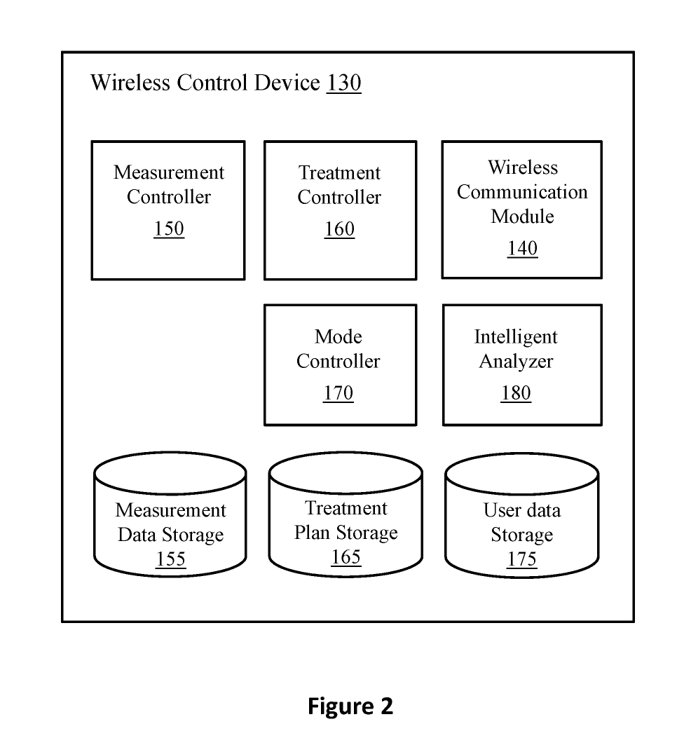

The wireless control device 130, referring to FIG. 2, includes a wireless communication module 140 that can wirelessly communicate with the networked wearable patch (110 in FIG. 1) using above described wireless technologies. The wireless control device 130 includes a measurement controller 150 that controls the wireless communication module 140 to transmit measurement control signals to the wearable patch (110 in FIG. 1). The measurement controller 150 can vary parameters of the measurements by the wearable patch. Such measurement parameters can include types, timing, frequencies, durations of measurements, coordination between measurements of the same or different wearable patches, and coordination between measurements and treatments by the wearable patch. A measurement data storage 155 stores the measurement data obtained by the wearable patch (110 in FIG. 1).

The wireless control device 130 includes a treatment controller 160 that can control the treatment functions of the wearable patch based on a treatment plan stored in the treatment plan storage 165. The treatment controller 160 can control the wireless communication module 140 to transmit treatment control signals to wearable patch (110 in FIG. 1). A treatment plan can define types, timing, frequencies, durations of treatments, coordination between treatments of the same of different wearable patch, and coordination between treatments and measurements by the wearable patch. A mode controller 170 can set the wearable patch in measurement modes and/or treatment modes, or a combination thereof by controlling the treatment unit and the sensors. Some treatment and measurements can be conducted in parallel, but some should be implemented at separate time periods. In some applications, a portion of the wearable patch (110 in FIG. 1) applies treatments while another portion of the wearable patch (110 in FIG. 1) conducts measurements. The coordination between measurement and treatment modes is controlled by the mode controller 170.

A user data storage 175 stores user data such as user's weight, height, bone density, historic range for blood pressure, heart beat, body temperature, daily patterns of exercises and rests by the user, sickness or symptoms suffered by the user, etc. In some embodiments, as described below, personalized medical treatment can be applied, sometimes dynamically, based on such user data.

An intelligent analyzer 180 can process and analyze the measurement data from different wearable patch in reference to the user data (in 175) and treatment plan (in 165) for the user. Using the measurement data and optionally historic user data, the intelligent analyzer 180 identifies improvement, issues, and risks in the user based on the measurement data to generate an analysis result, which could lead to timely reporting to the user or a central server, timely treatment, and/or improvement in the existing treatment. A portion of the analysis functions can be accomplished by a network server in communication with the wireless control device 130.

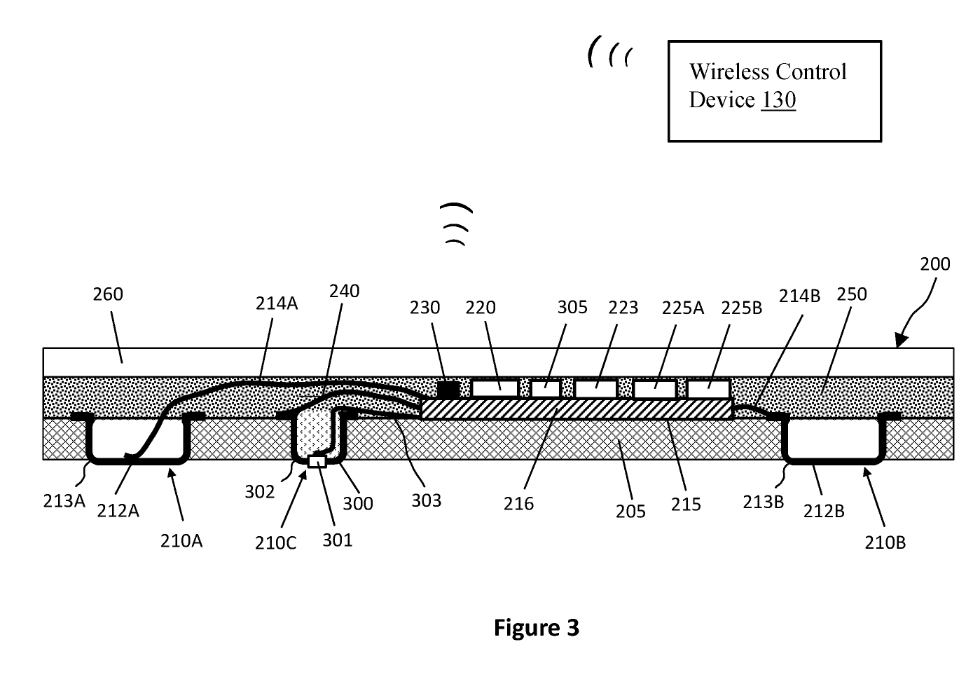

In some embodiments, referring to FIGS. 2-3, an exemplified wearable patch 200, which is suitable for the wearable patch 110 (FIG. 1), includes a stretchable substrate 205 that includes openings 210A, 210B, 210C. The stretchable substrate 205 can be made of soft foam materials such as EVA, PE, CR, PORON, EPD, SCF or fabric textile, to provide stretchability and breathability.

A circuit substrate 216 is mounted on the stretchable substrate 205 by an adhesive layer 215 pre-laminated on the stretchable substrate 205. The circuit substrate 216 includes an electric circuit therein and, for example, can be implemented with a printed circuit board.

A semiconductor chip 220, a memory 223, rechargeable batteries 225A, 225B, and an antenna 230 are mounted on the circuit substrate 216. Under the control of the semiconductor chip 220, the antenna 230 can wirelessly communicate with the wireless control device 130. Measurement control signals are received from the measurement controller 150 to control the measurements conducted by the pair of electrodes and the sensors in the wearable patch 110. The measurement data is transmitted to the wireless control device 130 to store in the measurement data storage 155 and analyzed by the intelligent analyzer 180 in the wireless control device 130.

The memory 223 stores long term ECG and measurement data before and even after the data is transmitted to the wireless control device 130. The rechargeable batteries 225A, 225B supply power to the electric circuit and the electronic components described above. One of the rechargeable batteries 225A, 225B can be separately removed and charged at a charging station while the other supplying power to enable continuous measurements and communication functions.

The wearable patch 200 includes electrodes 212A, 212B for measuring electrical signals from the user's body for continuous ECG recording. The electrodes 212A, 212B respectively includes electric conductive cups 213A, 213B respectively mounted in the openings 210A, 210B. The electric conductive cups 213A, 213B are respectively electrically connected to the electric circuit in the circuit substrate 216 by conductive lines 214A, 214B (e.g. flexible ribbons embedded with conductive circuits). The ECG signal (voltage) can be measured across two of the electrodes or across one of the electrodes and one of the electrodes 212A, 212B. The electrodes 212A, 212B can also be implemented in other configurations such as conductive pins, conductive pads, conductive buttons, or conductive strips.

The wearable patch 200 includes a third electrode 300 mounted in the opening 210C used for grounding purpose. The third electrode 300 can include an electric conductive cup 302, which is electrically connected with the circuit substrate 216 by a conductive line 240. The electrodes 212A, 212B and the third electrode 300 are configured to be in contact with a user's skin when the wearable patch 200 is worn by the user.

A mechanical sensor 301 is mounted in a window at the bottom of the cup 302 for detecting a pressure or a vibration in the user skin or body when the bottom of the cup 302 is in contact of the user's skin. The mechanical sensor 301 is electrically connected to the electric circuit in the circuit substrate 216 by a flexible conductive ribbon 303. The mechanical sensor 301 can include a piezoelectric material that produce electrical signal in response to pressure or stress. In one implementation, the mechanical sensor 301 can include a membrane coated with a piezoelectric material that produces an electrical signal in response to pressure, mechanical disturbances, or vibrations. In some implementations, the mechanical sensor 301 is an integrated micromechanical electrical system (MEMS) device that can be micro-fabricated on a semiconductor substrate. When the mechanical sensor 301 is in contact with user's skin, the vibrations or pressure variations caused by the user's heartbeats and blood pressure can be detected. The mechanical sensor 301 sends a measurement signal to the semiconductor chip 220 via a conductive line 303. Once the measurement data is received from the semiconductor chip 420 and the antenna 430, the measurement controller 150 and the intelligent analyzer 180 in the wireless control device 130 can extract the user's pulse and blood pressure information from the measurement data.

An acceleration sensor 305 is mounted on the circuit substrate 216 and in communication with the semiconductor chip 220. The acceleration sensor 305 can measure the movements of the user wearing the wearable patch 200. The ECG recording and blood pressure and acceleration measurements are under the control of the measurement controller 150 in the wireless control device 130.

In some embodiments, user's movement and pulse measurement data are used to determine the user's activity status. The ECG recording is continuously saved in the memory 223 on the wearable patch 200. Based on selected setting, there is automatic data transmission via the wireless control device 130 to the physician, or the patient can initiate data transmission when they feel needed. The wearable patch 200 powered by redundant rechargeable batteries 225A, 225B which allow battery charging and extended period of ECG recording.

The disclosed smart telehealth ECG recording system 100 (FIG. 1) can provide 1) smart options of ECG recordings with adequate retrospective data without worrying about missing any information, 2) convenient and continuous ambulatory recording, 3) prompt data sharing and analysis, as well as 4) extended recording time.

The physician may set automatic transmission conditions, for example, when the detected heart rate (not during activity) is above X bpm for Y seconds, or the detected heart rate during moderate exercise is above XX bpm for YY seconds for Z minutes of recordings. The physician can also set the patient initiated recordings, such as M min before the trigger and N min after the trigger. Extended pre-trigger recording durations are available with limited size of memory storage.

Measurement data from the acceleration sensor 305 can be used to categorize ECGs during rest or activity so that the analysis criteria can be used accordingly.

The patient can initiate a recording from the smart device when they feel symptoms or necessary. Since there is pre-trigger part of recording (which can be set very long), the user does not need to wait for the ECG to settle or worry about missing data that is before they feel the symptoms.

When the ECG recordings are transmitted to the smart device and then to the cloud, the physician or technician will be notified. It's their decision to do further analysis on the data or to review the data and send feedback or guidance of action back to the patient.

For example, a response treatment can be adjusted based on the measured user pulse and activity level. In some embodiments, measurement data obtained by the electrodes 212A, 212B are analyzed by the intelligent analyzer 180 to product an analysis result. Based on the analysis result, the mode controller 170 can switch the wearable patch 200 into a treatment mode.

An elastic layer 250 is bonded to the stretchable substrate 205 by the adhesive layer 215 to the stretchable substrate 205, and formed on the circuit substrate 216, and the electrodes 212A, 212B and the third electrode 300. The elastic layer 250 can be formed by soft stretchable and permeable foam materials such as EVA, PE, CR, PORON, EPD, SCF, or fabric textile. A thin film 260 is formed on the elastic layer 250 for protection and cosmetic purposes.

The semiconductor chip 220 can communicate with the wireless control device 130 via the antenna 230 in wireless signals. For example, the semiconductor chip 220 can receive a treatment sequence from the wireless control device 130. The wireless signal can be based on using Wi-Fi, Bluetooth, Near Field Communication (NFC), and other wireless standards. The semiconductor chip 220 can general the treatment electric signals at durations, intervals, and amplitudes as defined in the treatment plan.

In usage, an adhesive material formed on the lower surface of the stretchable substrate 205 is attached the user's skin, so that the bottom of the electrodes 212A, 212B, and the third electrode 300 are in contact with a user's skin for accurate ECG and pulse measurements. When the wearable patch 200 is worn by a user, the antenna 230 is separated from the user's skin by the circuit substrate 216 and the stretchable substrate 205, which minimizes the impact of the user's body on the transmissions of wireless signals by the antenna 230. The semiconductor chip 220 receives measurement signals from the electrodes 212A, 212B, the mechanical sensor 301, and the acceleration sensor 305.

In some embodiments, the mode controller 170 can switch a networked wearable patch into a measurement mode in response to the types of treatment applied. Under the control of the measurement controller 150, the semiconductor chip 220 can control the type(s), the timing, and frequencies of the measurement(s) by the electrodes 212A, 212B, the mechanical sensor 301, and the acceleration sensor 305 based on the treatment or monitoring plan. For example, the frequencies, the durations and the type(s) of the measurement(s) can be varied to more accurately and more timely monitor the user's health conditions.

Since the disclosed wearable patch is worn by an individual person, the disclosed wearable patch is ideal for personalized medical treatment. Each monitoring and treatment plan stored in the wireless control device 130 can be individualized according to the person's needs.

Other details about wearable patches capable of performing measurement and charging functions are disclosed in commonly assigned U.S. patent application Ser. No. 15/423,585, titled “A wearable patch comprising three electrodes for measurement and charging”, filed Feb. 3, 2017, commonly assigned U.S. patent application Ser. No. 15/457,532, titled “Dual purpose wearable patch for measurement and treatment”, filed Mar. 13, 2017, and commonly assigned U.S. patent application Ser. No. 15/472,641, titled “Multi-purpose wearable patch for measurement and treatment”, filed Mar. 29, 2017. The disclosures in the above applications are incorporated herein by reference.

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

Only a few examples and implementations are described. Other implementations, variations, modifications and enhancements to the described examples and implementations may be made without deviating from the spirit of the present invention.

What is claimed is:

1. A smart telehealth ECG recording system, comprising:
a wearable patch configured to be worn on a user's skin, comprising:
a stretchable substrate;
a pair of electrodes mounted in the stretchable substrate and configured to measurement a voltage signal across the user's skin to produce ECG data;
a mechanical sensor configured to measure a pulse from the user's body to produce pulse data;
an acceleration sensor configured to measure a movement of the user to produce movement data;
an antenna over the stretchable substrate and in electric communications with the pair of electrodes, the mechanical sensor, and the acceleration sensor, wherein the antenna is configured to wirelessly transmit the ECG data, the pulse data, and the movement data associated with the user; and
a circuit electrically connected with the pair of electrodes, the mechanical sensor, the acceleration sensor, and the antenna; and
a wireless control device configured to wirelessly receive the ECG data, the pulse data, and the movement data from the antenna, wherein the ECG data is analyzed based on the pulse data and the movement data.

2. The smart telehealth ECG recording system of claim 1, wherein the wearable patch further comprises:
a semiconductor chip in connection with the circuit and configured to produce electric signals comprising the ECG data, the pulse data, and the movement data, which enables the antenna to wirelessly transmit the ECG data, the pulse data, and the movement data.

3. The smart telehealth ECG recording system of claim 2, wherein the wearable patch further comprises:
a circuit substrate comprising the circuit and on the stretchable substrate, wherein the semiconductor chip is mounted on the circuit substrate.

4. The smart telehealth ECG recording system of claim 3, wherein the wireless control device comprises a measurement controller configured to wirelessly transmit a measurement control signal to the antenna, wherein the semiconductor chip is configured to measure ECG data, the pulse data, and the movement data in response to the measurement control signal.

5. The smart telehealth ECG recording system of claim 4, wherein the measurement controller is configured to control the semiconductor chip to vary a type, timing, a frequency, or duration of the ECG measurement, the pulse measurement, and the movement measurement.

6. The smart telehealth ECG recording system of claim 2, further comprising:
two rechargeable batteries configured to supply power to the circuit and the semiconductor chip, wherein at least one of the two rechargeable batteries is removeable to be charged.

7. The smart telehealth ECG recording system of claim 1, wherein the wearable patch further comprises:
a third electrode configured to be in contact with the user's skin, wherein the third electrode is in connection with the circuit and is configured to provide grounding to the circuit.

8. The smart telehealth ECG recording system of claim 1, wherein the stretchable substrate comprises an opening, wherein at least one of the pair of electrode comprises an electric conductive cup that is connected to the circuit and mounted in the opening in the stretchable substrate.

9. The smart telehealth ECG recording system of claim 1, wherein the wireless control device further comprises an intelligent analyzer configured to analyze the ECG data based on the pulse data and the movement data to produce an analysis result.

10. The smart telehealth ECG recording system of claim 1, wherein the wearable patch further comprises:
an elastic layer formed on the stretchable substrate, the circuit substrate, and the pair of electrodes.

11. A wearable patch configured to be worn on a user's skin, comprising:
a stretchable substrate;
a pair of electrodes mounted in the stretchable substrate and configured to measurement a voltage signal across the user's skin to produce ECG data;
a mechanical sensor configured to measure a pulse from the user's body to produce pulse data;
an acceleration sensor configured to measure a movement of the user to produce movement data;
an antenna over the stretchable substrate and in electric communications with the pair of electrodes, the mechanical sensor, and the acceleration sensor, wherein the antenna is configured to wirelessly transmit the ECG data, the pulse data, and the movement data associated with the user to a wireless control device; and
a circuit electrically connected with the pair of electrodes, the mechanical sensor, the acceleration sensor, and the antenna.

12. The wearable patch of claim 11, further comprising:
a semiconductor chip in connection with the circuit and configured to produce electric signals comprising the ECG data, the pulse data, and the movement data, which enables the antenna to wirelessly transmit the ECG data, the pulse data, and the movement data.

13. The wearable patch of claim 12, further comprising:
a circuit substrate comprising the circuit and on the stretchable substrate, wherein the semiconductor chip is mounted on the circuit substrate.

14. The wearable patch of claim 12, further comprising:
two rechargeable batteries configured to supply power to the circuit and the semiconductor chip, wherein at least one of the two rechargeable batteries is removeable to be charged.

15. The wearable patch of claim 11, further comprising:
a third electrode configured to be in contact with the user's skin, wherein the third electrode is in connection with the circuit and is configured to provide grounding to the circuit.

16. The wearable patch of claim 11, wherein the stretchable substrate comprises an opening, wherein at least one of the pair of electrode comprises an electric conductive cup that is connected to the circuit and mounted in the opening in the stretchable substrate.

17. The wearable patch of claim 11, further comprising:
an elastic layer formed on the stretchable substrate, the circuit substrate, and the pair of electrodes.

18. The wearable patch of claim 11, wherein the ECG data is analyzed based on the pulse data and the movement data to produce an analysis result.
